(19) United States
(12) Patent Application Publication
(10) Pub. No.: US 2018/0187191 A1
(43) Pub. Date: Jul. 5, 2018

(54) Targeted-Delivery of Small Interference RNA Against Anthrax

(71) Applicant: Texas Tech University System, Lubbock, TX (US)

(72) Inventor: Mingtao Zeng, El Paso, TX (US)

(21) Appl. No.: 15/736,358

(22) PCT Filed: Jun. 15, 2016
(86) PCT No.: PCT/US2016/037575

§ 371 Date: Dec. 14, 2017

Related U.S. Application Data
(60) Provisional application No. 62/175,951, filed on Jun. 15, 2015.

(51) Int. Cl. C12N 15/113; A61P 31/04; A61K 47/64

(52) U.S. Cl. CPC C12N 15/113; C12N 2310/14; A61K 47/6415; A61P 31/04

(57) ABSTRACT

The present invention includes a composition for the targeted-delivery of small interference RNA against bacteria comprising: a detoxified bacterial protein toxin that comprises a highly positively charged region; and an siRNA that is specific to, and knocks-down expression of one or more genes related to one or more virulence factors of the bacteria, wherein the siRNA is bound to the highly positively charged region of the detoxified bacterial protein toxin.

### TECHNICAL FIELD OF THE INVENTION

The present invention relates in general to the field of treatments for anthrax, and more particularly, to a novel method and compositions for targeted-delivery of small interference RNA against anthrax.

### BACKGROUND OF THE INVENTION

Without limiting the scope of the invention, its background is described in connection with alternative treatments against Bacillus anthracis infection or anthrax toxin exposure.

Inhalational anthrax is a leading bioterrorist threat and is fatal when left untreated. An anthrax vaccine has been licensed for human use (AVA or Biothrax, Emergent Biosolutions, Rockville, Md.), but the required immunization schedule is complicated, requiring six doses over 18 months followed by annual booster vaccinations. Post-exposure treatment for inhalational anthrax includes 60-day antibiotic therapy with a one-dose vaccination of AVA shortly after exposure, however, this treatment is unreliable at later stages of infection when large amounts of anthrax toxins have been produced.

Bacillus anthracis is the etiological agent responsible for anthrax. B. anthracis is a gram-positive, rod-shaped bacterium capable of forming stable and easily dispersible spores that can be developed and used as a bioweapon. Alveolar macrophages will ingest the B. anthracis spores following exposure via inhalation and transport these spores to draining lymph nodes where they germinate and produce virulence factors: a poly-D-glutamic acid capsule surrounding the vegetative form of the bacterium and toxins.

### SUMMARY OF THE INVENTION

Anthrax is a serious disease caused by Bacillus anthracis, a bacterium that forms spores. Anthrax most commonly occurs in wild and domestic mammalian species; but can also occur in humans when they are exposed to infected animals or animal tissues, or when anthrax spores are dispersed as a bioterrorist weapon. The complicated immunization schedule with the licensed vaccine BioThrax calls for a new and easily administered anthrax vaccine. Since anthrax is a disease that rarely occurs naturally in humans, it is more realistic to develop a post exposure prophylaxis or therapy instead of mass immunization, as with the current vaccine. Following exposure, macrophages ingest anthrax spores and travel to the lymph node where these spores germinate. The B. anthracis bacteria are then released into the bloodstream and produce toxins that are key factors in the virulence of disease: protective antigen (PA), edema factor (EF), and lethal factor (LF). PA is the receptor binding toxin component that attaches to either of two host cell receptors: anthrax toxin receptor 1 (ANTXR1 or tumor endothelial marker 8/TEM8) and anthrax toxin receptor 2 (ANTXR2 or capillary morphogenesis protein 2/CMG2). After binding, PA is cleaved and the receptor-bound portions form a heptameric pore that binds EF or LF. The toxin complexes are endocytosed and delivered into the cytosol. The activities of LeTx and EdTx result in malfunction of the immune system, edema, shock, and death. It is shown herein that inhibition of ANTXR expression by RNA interference (RNAi) technology using specific anti-ANTXR small interference RNA (siRNA) prevents cytotoxicity of anthrax toxins. Thus, a detoxified anthrax toxin can be used as a delivery vehicle for anti-ANTXR siRNA.

In one embodiment, the present invention includes a composition for the targeted-delivery of small interference RNA against bacteria comprising: a detoxified bacterial protein toxin that comprises a highly positively charged region; and an siRNA that is specific to, and knocks-down expression of one or more genes related to one or more virulence factors of the bacteria, wherein the siRNA is bound to the highly positively charged region of the detoxified bacterial protein toxin. In one aspect, the bacteria is Bacillus anthraces. In another aspect, the composition is adapted for post exposure prophylaxis or therapy. In another aspect, the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2). In another aspect, the detoxified bacterial toxin is selected from an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria. In another aspect, the detoxified bacterial toxin is selected from an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3)(SEQ ID NO:3) in EF (residues 136 to 142) or LF (residues 147 to 153), are a fusion protein that further comprises an exogenous peptide In another aspect, the detoxified bacterial toxin is selected from an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3)(SEQ ID NO:3) in EF (residues 136 to 142) or LF (residues 147 to 153) from the bacteria, are a fusion protein that further comprises an exogenous peptide, and a protective antigen (PA). In another aspect, the detoxified bacterial toxin is selected from an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3)(SEQ ID NO:3) in EF (residues 136 to 142) or LF (residues 147 to 153) from the bacteria, are a fusion protein that further comprises an influenza antigen, and a protective antigen (PA). In another aspect, the highly positively charged region comprises 5, 6, 7, 8, 9, 10, 11, 12, 13, 14, 15 or more arginine, lysine, or histidine residues, or combinations thereof.

In another embodiment, the present invention includes method of making a therapy against a bacterial infection comprising: preparing a detoxified bacterial protein toxin that comprises a highly positively charged region; and binding to the detoxified bacterial protein toxin an siRNA that is specific to, and knocks-down expression of one or more genes related to one or more virulence factors of the bacteria, wherein the siRNA is bound to the highly positively charged region of the detoxified bacterial protein toxin, wherein the detoxified bacterial protein toxin delivers the siRNA to a host cell. In one aspect, the bacteria is Bacillus anthraces. In another aspect, the composition is adapted for post exposure prophylaxis or therapy. In another aspect, the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA). In another aspect, the highly positively charged region comprises 5, 6, 7, 8, 9, 10, 11, 12, 13, 14, 15 or more arginine, lysine, or histidine residues, or combinations thereof.

In yet another embodiment, the present invention includes a composition for the targeted-delivery of small interference RNA against Bacillus anthracis comprising: a detoxified bacterial protein toxin that is a fusion protein that further comprises a highly positively charged region; and an siRNA that is specific to, and knocks-down expression of one or more genes related to one or more virulence factors of the Bacillus anthracis, wherein the siRNA is bound to the highly positively charged region of the detoxified bacterial protein toxin. In one aspect, the composition is adapted for post exposure prophylaxis or therapy. In another aspect, the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA). In another aspect, the highly positively charged region comprises 5, 6, 7, 8, 9, 10, 11, 12, 13, 14, 15 or more arginine, lysine, or histidine residues, or combinations thereof.

In another embodiment, the present invention includes a method of treating a subject suspected of being infected with a pathogenic bacterial comprising: preparing a detoxified bacterial protein toxin that comprises a highly positively charged region; binding to the detoxified bacterial protein toxin an siRNA to form a protein-siRNA complex, wherein the siRNA is specific to, and knocks-down expression of one or more genes related to one or more virulence factors of the bacteria, wherein the siRNA is bound to the highly positively charged region of the detoxified bacterial protein toxin, wherein the detoxified bacterial protein toxin delivers the siRNA to a host cell; and providing the subject an effective amount of the protein-siRNA complex sufficient to treat the bacterial infection. In one aspect, the bacteria is Bacillus anthracis. In another aspect, the composition is adapted for post exposure prophylaxis or therapy. In another aspect, the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide. In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA). In another aspect, the detoxified bacterial toxin is selected from an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA). In another aspect, the highly positively charged region comprises 5, 6, 7, 8, 9, 10, 11, 12, 13, 14, 15 or more arginine, lysine, or histidine residues, or combinations thereof.

### DESCRIPTION OF THE DRAWINGS

For a more complete understanding of the features and advantages of the present invention, reference is now made to the detailed description of the invention along with the accompanying figures and in which:

FIGS. 1a and 1b are graphs that show the effect of a detoxified anthrax toxin system that delivers influenza antigens as a protective vaccine. Mice were intranasally immunized 3 times with EFn-3×M2e-HA2±PA, then challenged with FIG. 1a: influenza virus PR8, intranasal. FIG. 1b: B. anthracis Sterne Spores, subcutaneous.

FIGS. 2a and 2b shows Anthrax toxin receptor mRNA expression in macrophages as determined by RT-PCR. Total RNA was isolated from cells and Superscript III reverse transcriptase was used to synthesize cDNA. FIG. 2a: In Raw 264.7 cells, murine TEM8 (258 bp), CMG2 (364 bp), and GAPDH (239 bp) cDNAs were amplified by gradient PCR (55-68.5 C). FIG. 2b: In THP-1 cells, human TEM8 (256 bp), CMG2 (344 bp), and GAPDH (930 bp) cDNAs were amplified by gradient PCR (55-69.1 C).

FIGS. 3a to 3d shows the siRNA-targeted silencing of CMG2 and evaluation of anthrax LeTx toxicity. FIG. 3a: Raw264.7 cells were cultured in 24-well plates and treated as follows: 1) untransfected (−), 2) RNAiMAX alone (L), 3) siGFP 10 and 20 pmol, 4) si-mTEM8 10 and 20 pmol, and 4) si-mCMG2 10 and 20 pmol. Total RNAs from these cells were isolated after 48 hours and RT-PCR was performed to amplify mCMG2 and mGAPDH fragments. FIG. 3b: 24-96 hours post-transfection. Cells were treated with siRNAs at times 0 and at 48 hours post-transfection. FIG. 3c: Cells were cultured in 96-well plates and were transfected twice with 5 pmol siGFP or si-mCMG2, or mock-transfected, then challenged with anthrax LeTx 48 hours after that last transfection. Data were normalized to cell viability controls (no LeTx) in each experiment. The mean+standard deviation (S.D.) of four experiments, performed in triplicates, is shown for all groups. FIG. 3d: Anti-CMG2 or an isotype control antibodies were allowed to bind to Raw 264.7 cells prior to addition of LeTx. One-way ANOVA and Dunnett post-hoc comparisons were performed for b and d. *p<0.05, ** p<0.01

FIG. 4 is a graph that shows siRNA-targeted silencing of ANTXRs protects against LeTX in human macrophage-like cells. THP-1 cells were differentiated with 10 nmol PMA for 3 days, treated for 24 h with 3 pmol siRNAs then challenged with LeTx. Mean+SD of 3 experiments performed in replicates of 6 are shown.

FIG. 5 is a graph that shows the kinetics of AP-1 mRNA silencing. Raw264.7 cells were treated with 20 pmol siRNAs 1) once and collected after 2 days; 2) on days 0 and 2 and collected on day 4; or 3) on days 0, 2, and 4 and collected on day 6. AP-1 and GAPDH transcript expressions were analyzed by RT-PCR.

FIG. 6 shows the highlights of one example of a structure of the composition of the present invention, including RRRRRRRRR (SEQ ID NO:1) and VYYEIGLGGGRRRRRRRRR (SEQ ID NO:2).

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Anthrax and the need for an effective treatment. Anthrax is a disease resulting from infection by spores of the Gram-positive bacterium Bacillus anthracis, a Category A Select Agent as designated by Centers for Disease Control (CDC). The formation of spores protects B. anthracis, allowing it to remain dormant and survive harsh chemical and thermal stresses until the local environment becomes more suitable for growth [20]. The disease manifests itself in three ways, resulting from three separate modes of infection. The most common occurrence of anthrax results from cutaneous exposure, where B. anthracis infects the host through a cut or abrasion on the skin. Secondly, digestive anthrax occurs upon consumption of contaminated food products by gaining entry into the gut. The final, and by far most deadly form of anthrax, is pulmonary or inhalational anthrax [21]. Although there is a licensed anthrax vaccine (BioThrax™) available for public use in the USA [22], it is not realistic to have a national immunization program in place since anthrax is a naturally rare disease in humans; and the complicated vaccine regimen makes this approach unrealistic anyway. In order to develop a more effective anthrax vaccine, currently, anthrax toxin components, PA and detoxified EF and LF, have been used as the key antigens in the current anthrax vaccine and in next-generation anthrax vaccines [7, 9, 10, 23-32]. Administration of BioThrax™ in combination with antibiotic may also provide certain benefit for post-exposure prophylaxis [33, 34]. Nevertheless, anthrax remains an imminent threat because it can be intentionally introduced by bioterrorists targeting individuals or the masses [35]. A few antibiotics, such as ciprofloxacin, can be used in killing B. anthracis bacteria. However, antibiotics are effective only prior to the onset of symptoms resulting from anthrax septicemia and toxemia because the toxins remain active long after bacterial death. In addition, antibiotic-resistant B. anthracis strains may be generated by bioterrorists. Clearly, a different strategy to develop a new and effective treatment as proposed in this research is imperative, and targeting toxin entry pathways and downstream cell death pathways may prove a successful approach for prophylactic and post exposure treatment against anthrax. Although not actually evaluated by human challenge study, analysis of human cases of naturally occurred inhalation anthrax has shown that the estimated median time from exposure to onset of symptoms (incubation period) among untreated cases to be 9.9 days (7.7-13.1) for exposure to ID50 of B. anthracis spores [36]. With advancement of the earlier and rapid detection technology for B. anthracis spore exposure and in the environment [37] as well as intellectually information gathering (such as in biodefense), this incubation period gives us a sufficient time window for administration of our host-targeted siRNA therapeutics with possible high efficacy for treatment.

Anthrax, which is caused by the spore-forming bacterium Bacillus anthracis, is one of the major bio-threats to public health. Following exposure of B. anthracis spores, macrophages ingest anthrax spores and travel to the lymph node where these spores germinate. The B. anthracis bacteria are then released into the bloodstream and produce toxins that are key factors in the virulence of disease: protective antigen (PA), edema factor (EF), and lethal factor (LF). Combination of LF and PA or EF and PA are named anthrax lethal toxin (LeTx) and edema toxin (EdTx), respectively. PA is the receptor binding toxin component that attaches to either of two host cell receptors: anthrax toxin receptor 1 (ANTXR1 or tumor endothelial marker 8/TEM8) and anthrax toxin receptor 2 (ANTXR2 or capillary morphogenesis protein 2/CMG2). After binding, PA is cleaved and the receptor-bound portions form a heptameric pore that binds EF or LF. The toxin complexes are endocytosed and delivered into the cytosol. The activities of LeTx and EdTx result in malfunction of the immune system, edema, shock, and death [1, 2].

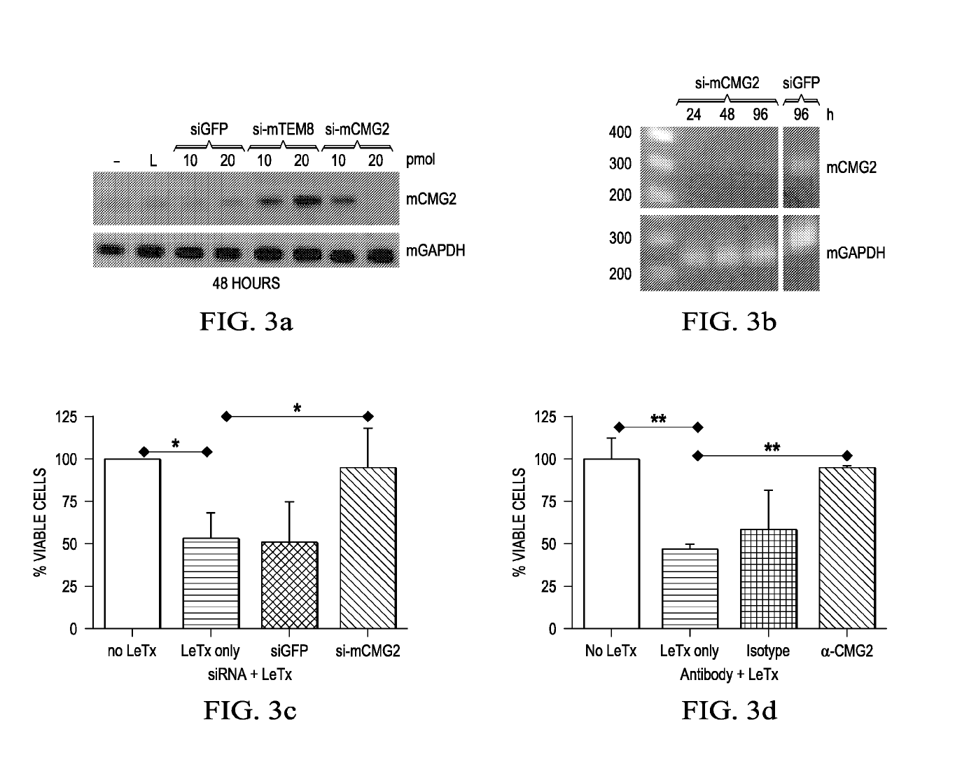

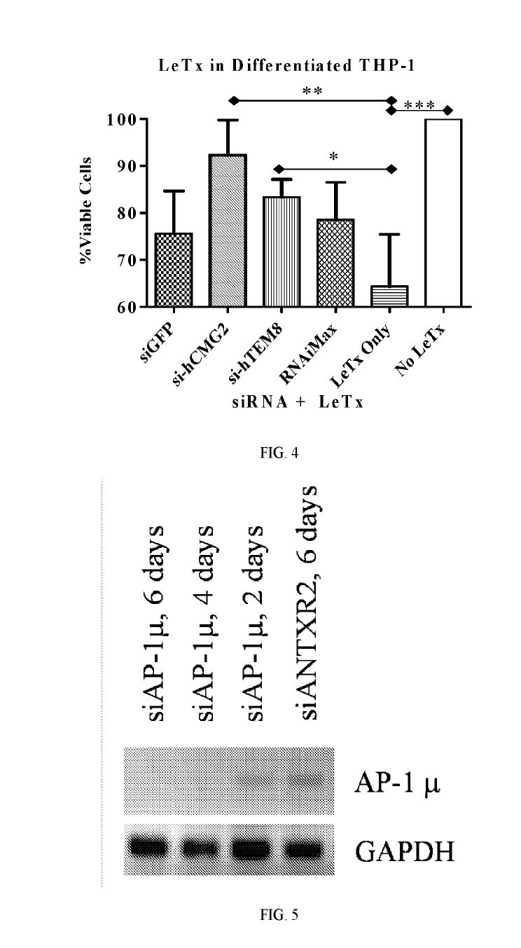

The current US human anthrax vaccine, BioThrax™, consists of aluminum hydroxide-adsorbed supernatant material, primarily protective antigen (PA) and undefined quantities of LF and EF, from fermentor cultures of a toxigenic, non-encapsulated strain of B. anthracis. Human vaccination with BioThrax™, requires six immunizations followed by annual boosters [3-5]. A relatively high local reaction rate of 3.6% in humans has been reported [6]. This underscores the need for development of new, improved anthrax vaccines. To date, there have been many attempts including research in the PI's lab to improve the safety profile and immunogenicity of the anthrax vaccine, including using multiple antigens [7-12]. However, none of the candidate vaccines is close to be licensed for public use in the near future [7, 8]. Since anthrax is a disease that rarely occurs naturally in humans, it is more realistic to develop a post exposure prophylaxis instead of mass immunization with the licensed vaccine. It is shown herein that inhibition of ANTXR expression by RNA interference (RNAi) technology using specific anti-ANTXR small interfering RNA (siRNA) prevents cytotoxicity of anthrax toxins (FIGS. 3-4) [13]. The novel host-targeted treatment shown herein against anthrax, is an example of a composition and method that can be used to overcome the weakness of the current antibiotic treatment in case of antibiotic resistant bacterial infection.

Thus, the present invention includes a novel host-targeted prophylactic or post exposure treatment against anthrax could be developed using a detoxified anthrax toxin as a delivery vehicle for anti-ANTXR siRNA.

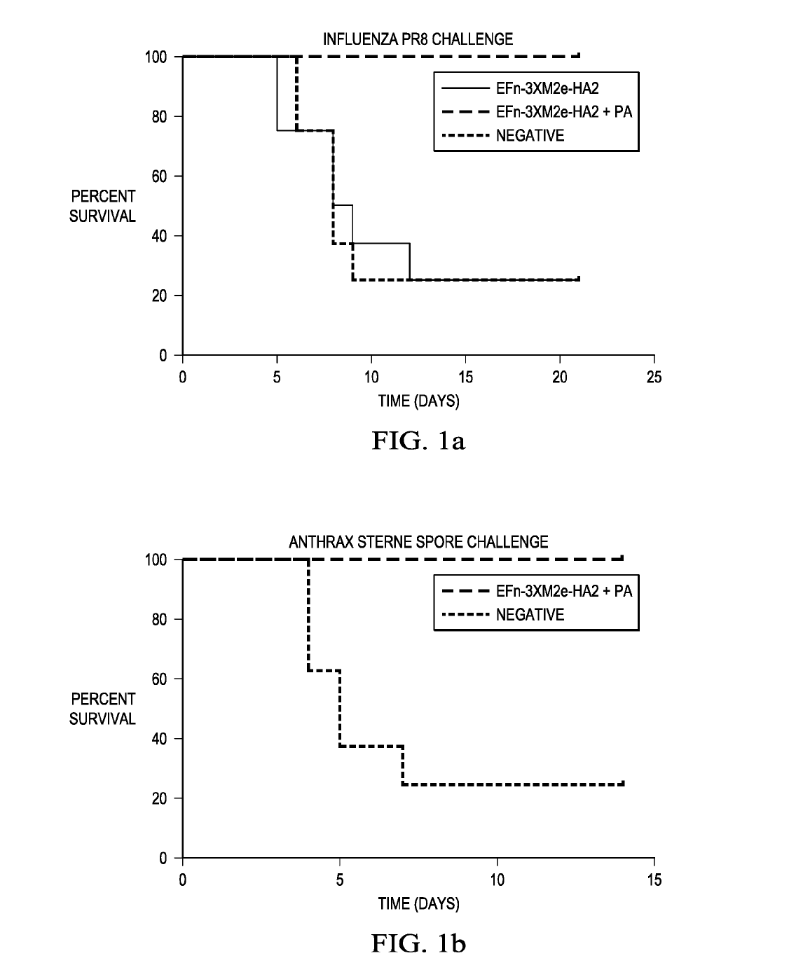

Generation of detoxified anthrax toxins for targeted siRNA delivery. The N-fragments of LF (EFn) and LF (LFn, the first 254 amino acids) are responsible for binding PA to translocate the enzymatic domain of the two toxin factors into the host cells and furnish the downstream toxicities. Since the EFn and LFn themselves are not toxic to cells, this property has been utilized to design a fusion protein to deliver foreign peptides into cells for vaccination [14, 15], relevant sequences incorporated herein by reference. The inventors have demonstrated that a detoxified EdTx (EFn fused with foreign peptide, plus PA) could be used for influenza antigen delivery to immune cells and generate protective immunity against both influenza and anthrax (FIGS. 1a to 1b) [12]. Thus, a detoxified anthrax toxin system can be used for delivery of cell target-specific therapeutic siRNA. A nontoxic EFn-9dR was generated that incorporates the PA binding domain (EFn) into a fusion protein with a highly positive 9 D-arginine residues (9dR) that enabled siRNA binding by charge interaction, which was shown by previous research [16, 17], relevant portions incorporated herein by reference.

To assess the inhibitory effect on anthrax toxin-induced cytotoxicity in ANTXR-silenced cells. The siRNA delivery will be first optimized with a GPF reporter system. EFn-9dR can be complexed with anti-GFP siRNA. Under the presence of PA63, EFn-9dR/siGFP is evaluated for silence of GFP expression in GFP stably transfected mouse macrophage like Raw 264.7 cells. Cy3-labeled siRNA can also be used to track the kinetics of siRNA delivery. Using the optimized condition, siRNA can be used to inhibit the expression of ANTXRs. Finally, cell survival following LeTx challenge can be evaluated first in mouse macrophages, then in human differentiated THP-1, smooth muscles, cardiomyocytes, and hepatocytes.

To evaluate efficacy of the anti-ANTXR siRNA treatment in a mouse model of anthrax. Selected siRNA formulations combined with detoxified anthrax toxins can be evaluated as prophylactic and post-exposure treatments against pulmonary anthrax. Intranasal inoculation of the complement-deficient A/J mouse with B. anthracis Sterne spores provides for a pulmonary anthrax model compatible with BSL2 containment [9, 10, 18, 19]. The protective efficacy of both respiratory and systemic deliveries of therapeutic siRNA can be evaluated. Co-administration of antibiotics can also be evaluated in comparison.

Target-specific RNAi is a safe and effective approach to treat severe infectious diseases. Despite recent advances in anti-pathogen approaches [38-44], host-side therapeutic intervention remains largely unexplored. RNA interference (RNAi) can be used to target several important host factors to block anthrax toxin endocytosis and the downstream activation of the inflammasome. This approach may work alone, or complement currently available antibiotic treatment for improved post-exposure prophylaxis of anthrax. RNAi is a recently discovered phenomenon in which small double-stranded RNAs (dsRNAs) regulate specific gene expression [45]. RNAi can be induced by either endogenously encoded small RNAs called microRNAs (miRNAs) or exogenously introduced small interfering RNAs (siRNAs). In either case, the 21-23 nucleotide dsRNAs associate in the cytoplasm with a protein complex called the RNA-induced silencing complex (RISC). One of the two RNA strands is degraded, and the other guiding strand guides the RISC to mediate the sequence-specific degradation of the corresponding mRNA (in the case of siRNAs) and/or translational repression by binding to the 3′ untranslated region (UTR) (in the case of miRNAs) [45]. The existence of RNAi machinery makes it possible for exotic designer small RNAs [synthetic siRNA or small hairpin RNA (shRNA)] to be used for silencing virtually any gene of interest in a sequence-specific manner. Ever since externally introduced double-stranded siRNAs were shown to silence specific gene expression in mammalian cells, there has been tremendous interest in using them as a research tool as well as applying them as novel drugs for the treatment of disease [46, 47]. RNAi may be useful in treating a variety of infectious diseases, including HIV, dengue, West Nile, St. Louis encephalitis, and respiratory syncytial virus (RSV) infections [48-54]. Furthermore, recent results from phase I clinical studies of siRNA targeting RSV nucleocapsid (N) protein as a treatment against RSV infection have demonstrated the safety and therapeutic potential of RNAi for human use [55-60]. Therefore, RNAi can readily be transformed to an effective therapeutic strategy in combating anthrax, a disease that could otherwise result in considerable morbidity and mortality even with antibiotic treatment [61].

RNAi approach. Building upon the present inventors previous work with RNAi technology and anthrax research [9, 10, 19, 48, 50-53, 62, 63], an RNAi strategy was developed to block anthrax toxin entry and signaling, thereby protecting the host from anthrax pathogenesis. This is a largely unexplored arena for effective treatment against anthrax.

Targeted siRNA delivery with a detoxified anthrax toxin. To mediate gene-silencing activity, intact double-stranded siRNAs have to be introduced into the cellular cytoplasm, where they can be recognized by the endogenous RNAi machinery and loaded onto RISC. The poor cellular uptake is the first major barrier for the use of siRNA, and limits its use even for local administration. The present invention uses an innovative detoxified anthrax toxin system for siRNA delivery to improve siRNA delivery in vivo. A detoxified anthrax toxin system can be used as a delivery vehicle into the host cell's cytosol [64]. Because only cells expressing anthrax toxins are directly involved in anthrax pathogenesis, the invention uses a detoxified anthrax toxin for therapeutic siRNA delivery. In this way, the molecular drug is specifically delivered to toxin receptor-expressing cells. This innovative approach selectively targets cell types or tissues involved in anthrax pathogenesis. To do so, the PA binding region EFn or its shorter peptide EF136-142 with a highly positive 9 D-arginine residues (9dR) that can bind negatively charged siRNA. The recombinant EFn-9dR or EF136-142-9dR plus PA can be specifically deliver siRNA to cells expressing anthrax toxin receptors. This dramatically increases the efficacy of RNAi and reduces potential side effects to cells not involved in anthrax pathogenesis.

The ANTXR-targeted RNAi approach may be used alone or in combination with available antibiotic treatments to improve anthrax post-exposure therapy. In addition, the non-antibiotic dependent host-targeted strategy can be used for effective therapy against anthrax caused by antibiotic resistant B. anthracis strains. Thus, the targeted-delivery of host-targeted siRNA will not only provide an effective prophylaxis for anthrax, but also a readily applicable treatment platform for other bacterial toxin-driven diseases.

Generate detoxified anthrax toxins for ANTXR-targeted siRNA delivery. FIGS. 1a and 1b shows the effects of a detoxified anthrax toxin system delivers influenza antigens as a protective vaccine. Mice were intranasally immunized 3 times with EFn-3×M2e-HA2±PA, then challenged with FIG. 1a: influenza virus PR8, intranasal. FIG. 1b: B. anthracis Sterne Spores, subcutaneous.

These studies show that a detoxified anthrax toxin system can not only be used for cell-targeted delivery of molecular therapies but also as an anthrax vaccine. An EFn-9dR for siRNA delivery was produced. 1) First, a fusion detoxified EF: EFn-Cys that has one additional cysteine at the C-terminal of EFn was made, which incorporates the PA binding domain (N-fragment of anthrax edema factor, the first 254 amino acids). Synthesized E. coli codon-optimized genes will be cloned into a prokaryotic expression vector pET200/D-TOPO® (provided by Invitrogen). 6×His tagged EFn-cys will be produced using BL21 Star™ (DE3) E. coli strain under the induction of Isopropyl-β-D-thiogalactopyranosid (IPTG). (Please note that there is no cysteine residue in the native sequence of EFn); 2) The recombinant EFn-cys produced in E. coli will be conjugated to a synthesized and modified peptide 9dR: Cys(Npys)-(D-Arg)9, to be purchased from Anaspec, CA (Cat #61206), using conjugation method described by our team previously [50]. The produced final EFn-9dR will be dialyzed and used in anti-ANTXR siRNA delivery in combination with PA.

It is also possible to use a minimized, detoxified anthrax toxin system for cell-targeted siRNA delivery. An EF136-142-9dR peptide construct can also be synthesized. The shorter, EF136-142 peptide is the PA-binding region of EF, and is identical to the PA binding domain in LF [65]. The synthetic EF136-142-9dR plus cleaved anthrax protective antigen (PA63) can specifically deliver siRNA to those cells expressing ANTXRs.

In case a prolonged expression of siRNA is required, we plan to use shRNA plasmid vector (The BLOCK-iT™ U6 RNAi Entry Plasmid Vector from Life Technologies). The EFn-9dR can be bound to the shRNA plasmid for intracellular delivery. Alternatively, a highly positive charged protamine can be added to EFn, and use the EFn-protamine for shRNA plasmid delivery, since the inventor's recent research has shown that a peptide-protamine system is highly efficient for plasmid DNA delivery in vivo [66]. In this way, smaller doses of the siRNA may be required for effective treatment.

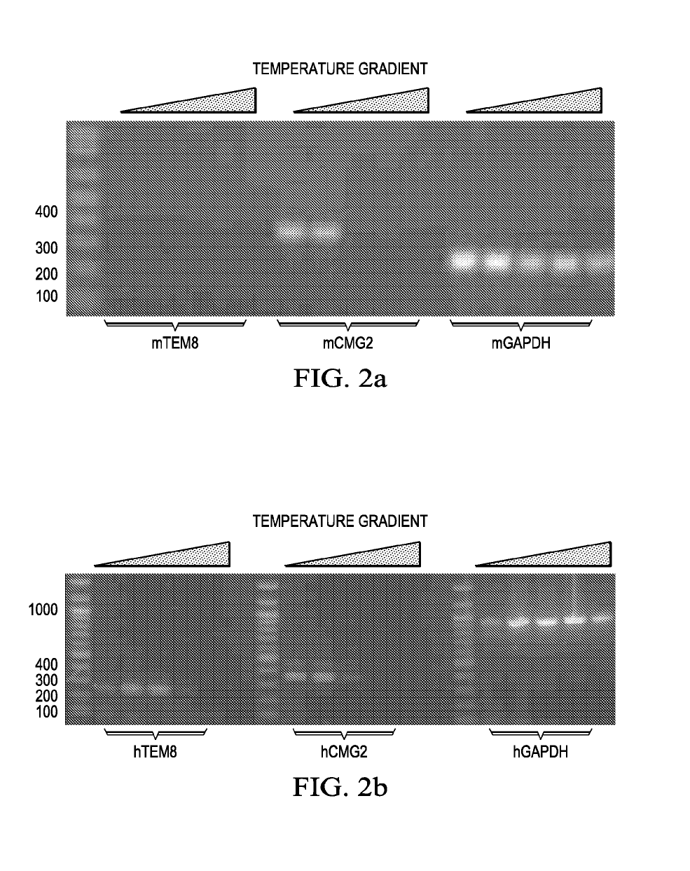

The inhibitory effect on toxin-induced cytotoxicity in ANTXR-silenced cells: silencing of anthrax toxin receptors. FIGS. 2a and 2b shows Anthrax toxin receptor mRNA expression in macrophages as determined by RT-PCR. Total RNA was isolated from cells and Superscript III reverse transcriptase was used to synthesize cDNA. FIG. 2a: In Raw 264.7 cells, murine TEM8 (258 bp), CMG2 (364 bp), and GAPDH (239 bp) cDNAs were amplified by gradient PCR (55-68.5 C). FIG. 2b: In THP-1 cells, human TEM8 (256 bp), CMG2 (344 bp), and GAPDH (930 bp) cDNAs were amplified by gradient PCR (55-69.1 C).

Optimization of siRNA-mediated silencing of ANTXR2 in Raw 264.7 cells. The silencing of ANTXR2/CMG2 transcript expression was optimized in Raw 265.7 cells cultured in 24-well culture plates since CMG2 is the dominant ANTXR in these cells. Cells were mock-transfected or transfected with RNAiMAX reagent (Life Technologies, NY), siRNA for GFP (siGFP) or siRNA for murine TEM8 (si-mTEM8) as non-specific siRNA controls, and siRNA for murine CMG2 (si-mCMG2) (reported recently [13]). RT-PCR analyses revealed that 20 pmol of si-CMG2 specifically and effectively silenced CMG2 mRNA expression at 48 hours post transfection (FIG. 3a) GAPDH transcript expression was monitored in parallel as an additional control (FIG. 3a). Next, we assessed CMG2 transcript expression in Raw 264.7 cell treated with 20 pmol of si-mCMG2 from 24-96 h post-transfection. Cells were treated with siRNAs at times 0 and at 48 h post-transfection. RT-PCR analysis verified that CMG2 transcript expression was silenced during this time period while levels of GAPDH transcript remained stable (FIG. 3b).

Evaluation of anthrax LeTx-mediated toxicity in anthrax receptor-silenced Raw 264.7 cells. Since CMG2 is the dominant ANTXR in Raw 264.7 cells, it was determined if CMG2-silenced Raw 264.7 cells were protected against LeTx under the conditions described above. Cell viability was assessed following 7-hour LeTx treatments using a colorimetric, MTT assay in 96-well plates (FIG. 3c). After two treatments, protection against LeTx in cells that were treated with CMG2-specific siRNAs over untreated cells was observed.

FIGS. 3a to 3d shows the siRNA-targeted silencing of CMG2 and evaluation of anthrax LeTx toxicity. FIG. 3a: Raw264.7 cells were cultured in 24-well plates and treated as follows: 1) untransfected (−), 2) RNAiMAX alone (L), 3) siGFP 10 and 20 pmol, 4) si-mTEM8 10 and 20 pmol, and 4) si-mCMG2 10 and 20 pmol. Total RNAs from these cells were isolated after 48 hours and RT-PCR was performed to amplify mCMG2 and mGAPDH fragments. FIG. 3b: 24-96 hours post-transfection. Cells were treated with siRNAs at times 0 and at 48 hours post-transfection. FIG. 3c: Cells were cultured in 96-well plates and were transfected twice with 5 pmol siGFP or si-mCMG2, or mock-transfected, then challenged with anthrax LeTx 48 hours after that last transfection. Data were normalized to cell viability controls (no LeTx) in each experiment. The mean+standard deviation (S.D.) of four experiments, performed in triplicates, is shown for all groups. FIG. 3d: Anti-CMG2 or an isotype control antibodies were allowed to bind to Raw 264.7 cells prior to addition of LeTx. One-way ANOVA and Dunnett post-hoc comparisons were performed for b and d. *p<0.05, ** p<0.01

FIG. 4 is a graph that shows that siRNA-targeted silencing of ANTXRs protects against LeTX in human macrophage-like cells. THP-1 cells were differentiated with 10 nmol PMA for 3 days, treated for 24 h with 3 pmol siRNAs then challenged with LeTx. Mean+SD of 3 experiments performed in replicates of 6 are shown.

These data show that RNAi-mediated silencing of ANTXRs is a potent strategy against anthrax toxin-induced death in cultured mouse and human macrophages, maintaining cell viability above 92% as compared to appropriate controls.

Optimization of siRNA delivery in vitro. EFn-9dR, or EF136-142-9dR can be first complexed with anti-GFP siRNA. Under the presence of PA63, the complexes will be evaluated for silencing GFP expression in GFP stably transfected Raw 264.7 cells, which have been generated in our Lab (Data not shown). A fluorophore-labeled (Cy3) siRNA can be used (GE Dharmacon) to track the process of siRNA delivered with detoxified anthrax toxin. This will elucidate the kinetics of our siRNA in terms of cell internalization and receptor binding affinity. Under the optimized condition, it is possible to use the optimal siRNA/detoxified toxin formulation to inhibit the expression of CMG2 in Raw 264.7 cells, since CMG2 is the dominant ANTXR in these cells. Cell survival following anthrax lethal toxin (LeTx) challenge will be evaluated.

An evaluation for TEM8-targeted siRNAs/detoxified toxin formulation in TEM8 expressing cells, such as human umbilical vein endothelial cells (HUVEC) [69] can also be conducted. Additionally, differentiated human THP-1 cells, cultured primary mouse and human macrophages will be evaluated using combined siRNA/detoxified toxin formulation for both TEM8 and CMG2.

Furthermore, the present invention can be used to test protection in vitro by these siRNAs/detoxified toxin formulation in primary human smooth muscle cells, cardiomyocytes, and hepatocytes—because these cells are essential in anthrax toxin-induced lethality as shown in mice [70]. The present invention is also useful for screening of super-high potent siRNAs for targeting host factors in anthrax pathogenesis. If the cytotoxicity of any of the siRNAs is unacceptable (e.g., 10% cell death) or do not show an acceptable protection (50%) against LeTx-induced cell death, we will redesign siRNAs using our unique super-high potent siRNAs screening method. It is feasible to screen out therapeutic siRNAs with IC50 of less than 10 pM.

Finally, aside from cell death, the present invention can be used to monitor the effects of the anthrax toxins by tracking signaling processes resulting from their introduction into the cytosol. Anthrax lethal toxin cleaves mitogen-activated protein kinase kinase (MAPKK) [71, 72], activates the NOD-like receptor NLRP1 inflammasome [73-75], and causes cell death while edema toxin leads to increased cellular cyclic AMP and consequent swelling [76]. We will test if silencing TEM8 and CMG2 blocks MAPKK cleavage, activates the NLRP1 inflammasome, and increases in cellular cAMP, using methods described previously by the inventor and others [10, 13, 74, 77, 78].

To evaluate efficacy of the anti-ANTXR siRNA treatment in a mouse model of anthrax. The efficacy of both prophylactic and post-exposure treatments with our formulated siRNA can be tested. Since the complement-deficient A/J mouse is susceptible to the attenuated non-capsular, toxigenic B. anthracis Sterne strain, it is possible to evaluate our therapeutic approach using a pulmonary anthrax model induced by intranasal inoculation of B. anthracis Sterne spores under BSL2 containment [18]. This mouse anthrax model has been successfully used for vaccine evaluation by the present inventors [9-11, 19]. This model can be used to evaluate selected single and combinatory siRNA treatments after pulmonary exposure to spores. The invention can also be used to determine the protective efficacy of: 1) formulated siRNA or shRNA plasmid with detoxified anthrax toxin; 2) siRNA formulated with drug carriers such as poly (lactic-co-glycolic acid) (PLGA) or chitosan nanoparticles; 3) respiratory endotracheal delivery in comparison with systemic intravenous (IV) delivery; 4) siRNA treatments in combination with antibiotic administration.

Efficacy of prophylactic treatment against B. anthracis spore challenge. Three days before spore challenge, A/J Mice will be first treated with detoxified anthrax toxins combining single and multiple siRNA or shRNA plasmid formulations (CMG2, TEM8, and their combination) for endotracheal administration to ensure that the formulated siRNA will reach the lung. In comparison, we will test systemic IV delivery of formulated siRNA to ensure siRNA will reach all target cells. As a control, commercially available polymer-based reagent in vivo-JetPEI (from Polyplus-Transfection) will also be used for siRNA delivery as previously described [79]. A daily 5 nmol siRNA/dose will be initially used. For comparative studies, Ciprofloxacin antibiotic treatment (twice daily, at 30 mg/kg, by the intraperitoneal route) will also be evaluated [80]. As an additional control, the FDA approved anti-PA human mAb (Raxibacumab) will be used [81]. Table 1 lists the groups for study.

TABLE 1
Groups for initial prophylactic and post-exposure treatment
with siRNA (8 mice/group).

| | | | Post- | | Post |
|---|---|---|---|---|---|
| | Prophylactic | | exposure | | exposure |
| Group | Treatment | Group | Treatment | Group | Treatment |
| 1 | EFn-9dR- | 7 | EFn-9dR- | 13 | Control: |
| | siCMG2/PA | | siCMG2/PA | | placebo |
| 2 | EFn-9dR- | 8 | EFn-9dR- | 14 | Control: |
| | siTEM8/PA | | siTEM8/PA | | antibiotic |
| | | | | | Ciprofloxacin |
| 3 | EFn-9dR- | 9 | EFn-9dR- | 15 | Control: |
| | siCMG2 & | | siCMG2 & | | Anti-PA |
| | EFn-9dR- | | EFn-9dR- | | Raxibacumab |
| | siTEM8/PA | | siTEM8/PA | | |
| 4 | siCMG2/in | 10 | siCMG2/in | | |
| | vivo-JetPEI | | vivo-JetPEI | | |
| 5 | siTEM8/in | 11 | siTEM8/in | | |
| | vivo-JetPEI | | vivo-JetPEI | | |
| 6 | siCMG2 & | 12 | siCMG2 & | | |
| | siTEM8/in | | siTEM8/in | | |
| | vivo-JetPEI | | vivo-JetPEI | | |

Other combinatorial approaches can also be used, such as, a combination of siRNA and antibiotic daily administration. From a statistical power analysis, a group size is 8 mice per group can be used. Mice can be inoculated with lethal doses of B. anthracis Sterne spores by the intranasal route. Experimental procedures include administration of 10-100×MLD50 B. anthracis Sterne spore by the intranasal route, and administration of RNAi and/or antibiotics daily will continue for 2 weeks after spore challenge. Disease severity and death will be carefully monitored. At the end of the experiment, mice that survived the treatment can be sacrificed and their internal organs such as lungs, liver, spleen, and kidneys, will be collected for histological analysis by our pathologist from histopathology core.

Efficacy of post exposure treatment against B. anthracis spore challenge. Similarly, A/J Mice can be inoculated with lethal doses of B. anthracis Sterne spores by the intranasal route. One day later, detoxified anthrax toxins combining with single and multiple siRNA or shRNA plasmid formulations (CMG2, TEM8, and their combination) can be administered endotracheally or intravenously. Ciprofloxacin antibiotic and anti-PA treatments will be used as treatment control (See Table 1 for initial groups). Again, mice can be treated for 2 weeks. Survival data and histopathological analysis can be done at the end of the experiment. After the initial study, the siRNA treatment time and doses can be further optimization.

Evaluation of effects of silencing ANTXRs. CMG2 and TEM8 are ubiquitously expressed receptors with roles in regulation of matrix metalloproteinase activity and extracellular matrix (ECM) homeostasis [92]. TEM8 or CMG2 null or knock-out mice are viable, develop normally, and survive into adulthood [93-96]. Phenotypically, the main defect in these mice is excessive ECM protein deposition [93, 96]. Aberrant ECM deposition in the cervix and uterus of females has been associated with fertility defects observed in the females of null or knockout mice [96]. Thus, a possible side effect of silencing ANTXRs would be increased ECM deposition for the transient period in which these receptors are silenced. This would be a minor and transient inconvenience, not comparable to the pathogenesis and lethality of anthrax disease caused by anthrax toxins. ECM deposition in ANTXR-silenced mice can be determined by histology using Masson's Trichrome stain [96] of tissue sections. One of the solutions, the fibre stain, stains collagen green or blue.

An additional advantage of the proposed targeted-siRNA delivery strategy is that, currently, anthrax toxin components, PA and detoxified EF and LF, have been demonstrated by the present inventor and others as promising vaccine antigens for next-generation anthrax vaccines [7, 9, 10, 23-32]. The proposed targeted siRNA delivery system can also be developed as a post-exposure therapeutic vaccine against anthrax.

FIG. 5 is a graph that shows the kinetics of AP-1 mRNA silencing. Raw264.7 cells were treated with 20 pmol siRNAs 1) once and collected after 2 days; 2) on days 0 and 2 and collected on day 4; or 3) on days 0, 2, and 4 and collected on day 6. AP-1 and GAPDH transcript expressions were analyzed by RT-PCR.

FIG. 6 shows the highlights of one example of a structure of the composition of the present invention, including RRRRRRRRR (SEQ ID NO:1) and VYYEIGLGGGRRRRRRRRR (SEQ ID NO:2. Briefly, (1) EFn/LFn-C=C-RRRRRRRRR (SEQ ID NO:1) can be replaced by the PA binding peptide from EF or LF fused with the positively charged tail: VYYEIGL-GGGRRRRRRRRR (SEQ ID NO: (2)), where R is D-Arginine (D-Arg).

Statistical Analysis. Based on previously published studies and preliminary research, animal groups for each experiment will contain at least 8 mice. To compare the average response across the groups used in each study it is possible to perform two-tailed ANOVA analysis followed by adjusted post-hoc t-tests. In addition, if warranted due to skewed data distributions, a non-parametric statistical method, such as the Kruskal-Wallis test, can be used. For analyses of time to event/death it is possible to use the Kaplan-Meier method of survivorship function estimation and associated graphical methods as well as the Log-rank test to compare survivorship across the groups used in each study.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure.

Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] Dixon T C, Meselson M, Guillemin J, Hanna P C. Anthrax. N Engl J Med. 1999; 341:815-26.
[2] Swartz M N. Recognition and management of anthrax—an update. N Engl J Med. 2001; 345:1621-6.
[3] Bienek D R, Loomis L J, Biagini R E. The anthrax vaccine: no new tricks for an old dog. Hum Vaccin. 2009; 5:184-9.
[4] EBS. BioThrax (R) Anthrax Vaccine Adsorbed Dosage and Administration.
[5] Friedlander A M, Pittman P R, Parker G W. Anthrax vaccine: evidence for safety and efficacy against inhalational anthrax. Jama. 1999; 282:2104-6.
[6] Pittman P R, Gibbs P H, Cannon T L, Friedlander A M. Anthrax vaccine: short-term safety experience in humans. Vaccine. 2001; 20:972-8.
[7] Chitlaru T, Altboum Z, Reuveny S, Shafferman A. Progress and novel strategies in vaccine development and treatment of anthrax. Immunol Rev. 2011; 239:221-36.
[8] Friedlander A M, Welkos S L, Ivins B E. Anthrax vaccines. Current topics in microbiology and immunology. 2002; 271:33-60.
[9] Xu Q, Zeng M. Detoxified Lethal Toxin as A Potential Mucosal Anthrax Vaccine. Clinical and Vaccine Immunology. 2008; 15:612-6. PMID: 18256208
[10] Zeng M, Xu Q, Hesek E D, Pichichero M E. N-fragment of edema factor as a candidate antigen for immunization against anthrax. Vaccine. 2006; 24:662-70 [Epub 2005 Aug. 26]. PMID: 16157430.
[11] Zeng M, Xu Q, Pichichero M E. Protection against anthrax by needle-free mucosal immunization with human anthrax vaccine. Vaccine. 2007; 25:3558-94 [Epub 2007 Jan. 26]. PMID: 17293013.
[12] Arevalo M T, Li J, Diaz-Arevalo D, Chen Y, Zeng M. A dual purpose universal influenza vaccine candidate confers protective immunity against anthrax. Submitted to Journal of Immunology (Under revision). 2014.
[13] Arevalo M T, Navarro A, Arico C D, Li J, Alkhatib O, Chen S, et al. Targeted Silencing of Anthrax Toxin Receptors Protects against Anthrax Toxins. The Journal of biological chemistry. 2014; 289:15730-8.
[14] McEvers K, Elrefaei M, Norris P, Deeks S, Martin J, Lu Y, et al. Modified anthrax fusion proteins deliver HIV antigens through MHC Class I and II pathways. Vaccine. 2005; 23:4128-35. Epub 2005 Apr. 15.
[15] Goletz T J, Klimpel K R, Arora N, Leppla S H, Keith J M, Berzofsky J A. Targeting HIV proteins to the major histocompatibility complex class I processing pathway with a novel gp120-anthrax toxin fusion protein. Proc Natl Acad Sci USA. 1997; 94:12059-64.
[16] Kumar P, Wu H, McBride J L, Jung K E, Kim M E I, Davidson B L, et al. Transvascular delivery of small interfering RNA to the central nervous system. Nature. 2007; 448:39-43.
[17] Ye C, Abraham S, Wu H, Shankar P, Manjunath N. Silencing early viral replication in macrophages and dendritic cells effectively suppresses flavivirus encephalitis. PLoS One. 2011; 6:e17889.
[18] Yadav J S, Pradhan S, Kapoor R, Bangar H, Burzynski B B, Prows D R, et al. Multigenic control and sex bias in host susceptibility to spore-induced pulmonary anthrax in mice. Infect Immun. 2011; 79:3204-15.
[19] Xu Q, Hesek E D, Zeng M. Transcriptional stimulation of anthrax toxin receptors by anthrax edema toxin and Bacillus anthracis Sterne spore. Microb Pathog. 2007; 43:37-45. PMID: 17459655.
[20] Baillie L, Hibbs S, Tsai P, Cao G L, Rosen G M. Role of superoxide in the germination of Bacillus anthracis endospores. FEMS Microbiol Lett. 2005; 245:33-8.
[21] Bradley K A, Young J A. Anthrax toxin receptor proteins. Biochem Pharmacol. 2003; 65:309-14.
[22] Anthrax Vaccine Adsorbed (BioThrax™) Product Insert. Lansing, Mich.: Bioport Corporation; 2002.
[23] Ivins B E, Welkos S L, Little S F, Crumrine M H, Nelson G O. Immunization against anthrax with Bacillus anthracis protective antigen combined with adjuvants. Infect Immun. 1992; 60:662-8.
[24] Matyas G R, Friedlander A M, Glenn G M, Little S, Yu J, Alving C R. Needle-free skin patch vaccination method for anthrax. Infect Immun. 2004; 72:1181-3.
[25] Kenney R T, Yu J, Guebre-Xabier M, Frech S A, Lambert A, Heller B A, et al. Induction of Protective Immunity against Lethal Anthrax Challenge with a Patch. J Infect Dis. 2004; 190:774-82. Epub 2004 Jul. 13.
[26] Singh Y, Ivins B E, Leppla S H. Study of immunization against anthrax with the purified recombinant protective antigen of Bacillus anthracis. Infect Immun. 1998; 66:3447-8.
[27] Flick-Smith H C, Walker N J, Gibson P, Bullifent H, Hayward S, Miller J, et al. A recombinant carboxy-terminal domain of the protective antigen of Bacillus anthracis protects mice against anthrax infection. Infect Immun. 2002; 70:1653-6.
[28] Pezard C, Weber M, Sirard J C, Berche P, Mock M. Protective immunity induced by Bacillus anthracis toxin-deficient strains. Infect Immun. 1995; 63:1369-72.
[29] Little S F. Anthrax vaccines: a development update. BioDrugs: clinical immunotherapeutics, biopharmaceuticals and gene therapy. 2005; 19:233-45.
[30] Price B M, Liner A L, Park S, Leppla S H, Mateczun A, Galloway D R. Protection against anthrax lethal toxin challenge by genetic immunization with a plasmid encoding the lethal factor protein. Infect Immun. 2001; 69:4509-15.
[31] Galloway D, Liner A, Legutki J, Mateczun A, Barnewall R, Estep J. Genetic immunization against anthrax. Vaccine. 2004; 22:1604-8.
[32] Oscherwitz J, Yu F, Cease K B. A synthetic peptide vaccine directed against the 2ss2-2ss3 loop of domain 2 of protective antigen protects rabbits from inhalation anthrax. J Immunol. 2010; 185:3661-8.
[33] Ionin B, Hopkins R J, Pleune B, Sivko G S, Reid F M, Clement K H, et al. Evaluation of immunogenicity and efficacy of anthrax vaccine adsorbed for postexposure prophylaxis. Clin Vaccine Immunol. 2013; 20:1016-26.
[34] Vietri N J, Purcell B K, Lawler J V, Leffel E K, Rico P, Gamble C S, et al. Short-course postexposure antibiotic prophylaxis combined with vaccination protects against experimental inhalational anthrax. Proc Natl Acad Sci USA. 2006; 103:7813-6.
[35] Inglesby T V, O'Toole T, Henderson D A, Bartlett J G, Ascher M S, Eitzen E, et al. Anthrax as a biological weapon, 2002: updated recommendations for management. Jama. 2002; 287:2236-52.
[36] Toth D J, Gundlapalli A V, Schell W A, Bulmahn K, Walton T E, Woods C W, et al.
Quantitative models of the dose-response and time course of inhalational anthrax in humans. PLoS pathogens. 2013; 9:e1003555.
[37] Irenge L M, Gala J L. Rapid detection methods for Bacillus anthracis in environmental samples: a review. Appl Microbiol Biotechnol. 2012; 93:1411-22.
[38] Mourez M. Anthrax toxins. Rev Physiol Biochem Pharmacol. 2004; 152:135-64. Epub 2004 Jul. 27.
[39] Karginov V A, Robinson™, Riemenschneider J, Golding B, Kennedy M, Shiloach J, et al. Treatment of anthrax infection with combination of ciprofloxacin and antibodies to protective antigen of Bacillus anthracis. FEMS Immunol Med Microbiol. 2004; 40:71-4.
[40] Bradley K A, Mogridge J, Mourez M, Collier R J, Young J A. Identification of the cellular receptor for anthrax toxin. Nature. 2001; 414:225-9.
[41] Scobie H M, Rainey G J, Bradley K A, Young J A. Human capillary morphogenesis protein 2 functions as an anthrax toxin receptor. Proc Natl Acad Sci USA. 2003; 100:5170-4.
[42] Tonello F, Seveso M, Marin O, Mock M, Montecucco C. Screening inhibitors of anthrax lethal factor. Nature. 2002; 418:386.
[43] Panchal R G, Hermone A R, Nguyen T L, Wong T Y, Schwarzenbacher R, Schmidt J, et al. Identification of small molecule inhibitors of anthrax lethal factor. Nat Struct Mol Biol. 2004; 11:67-72. Epub 2003 Dec. 29.
[44] Mourez M, Kane R S, Mogridge J, Metallo S, Deschatelets P, Sellman B R, et al. Designing a polyvalent inhibitor of anthrax toxin. Nat Biotechnol. 2001; 19:958-61.
[45] Kim S S, Garg H, Joshi A, Manjunath N. Strategies for targeted nonviral delivery of siRNAs in vivo. Trends Mol Med. 2009; 15:491-500.
[46] Castanotto D, Rossi J J. The promises and pitfalls of RNA-interference-based therapeutics. Nature. 2009; 457:426-33.
[47] Manjunath N, Kumar P, Lee S K, Shankar P. Interfering antiviral immunity: application, subversion, hope? Trends Immunol. 2006; 27:328-35.
[48] Subramanya S, Armant M, Salkowitz J R, Nyakeriga A M, Haridas V, Hasan M, et al. Enhanced induction of HIV-specific cytotoxic T lymphocytes by dendritic cell-targeted delivery of SOCS-1 siRNA. Mol Ther. 2010; 18:2028-37.
[49] Song E, Zhu P, Lee S K, Chowdhury D, Kussman S, Dykxhoorn D M, et al. Antibody mediated in vivo delivery of small interfering RNAs via cell-surface receptors. Nat Biotechnol. 2005; 23:709-17.
[50] Kumar P, Ban H S, Kim S S, Wu H, Pearson T, Greiner D L, et al. T cell-specific siRNA delivery suppresses HIV-1 infection in humanized mice. Cell. 2008; 134:577-86.
[51] Kim S S, Peer D, Kumar P, Subramanya S, Wu H, Asthana D, et al. RNAi-mediated CCRS silencing by LFA-1-targeted nanoparticles prevents HIV infection in BLT mice. Mol Ther. 2010; 18:370-6.
[52] Subramanya S, Kim S S, Abraham S, Yao J, Kumar M, Kumar P, et al. Targeted delivery of small interfering RNA to human dendritic cells to suppress dengue virus infection and associated proinflammatory cytokine production. J Virol. 2010; 84:2490-501.
[53] Wu H, Ma H, Ye C, Danielle Ramirezl, Chen S, Montoya J, et al. Improved siRNA/shRNA Functionality by Mismatched Duplex. PLoS ONE. 2011; 6:e28580. doi:10.1371/journal.pone.0028580.
[54] Alvarez R, Elbashir S, Borland T, Toudjarska I, Hadwiger P, John M, et al. RNA interference-mediated silencing of the respiratory syncytial virus nucleocapsid defines a potent antiviral strategy. Antimicrobial agents and chemotherapy. 2009; 53:3952-62.
[55] DeVincenzo J, Cehelsky J E, Alvarez R, Elbashir S, Harborth J, Toudjarska I, et al. Evaluation of the safety, tolerability and pharmacokinetics of ALN-RSV01, a novel RNAi antiviral therapeutic directed against respiratory syncytial virus (RSV). Antiviral research. 2008; 77:225-31.
[56] DeVincenzo J, Lambkin-Williams R, Wilkinson T, Cehelsky J, Nochur S, Walsh E, et al. A randomized, double-blind, placebo-controlled study of an RNAi-based therapy directed against respiratory syncytial virus. Proc Natl Acad Sci USA. 2010; 107:8800-5.
[57] Zamora M R, Budev M, Rolfe M, Gottlieb J, Humar A, Devincenzo J, et al. RNA interference therapy in lung transplant patients infected with respiratory syncytial virus. American journal of respiratory and critical care medicine. 2011; 183:531-8.
[58] Crunkhorn S. Trial watch: Success in amyloidosis trials supports potential of systemic RNAi. Nat Rev Drug Discov. 2013; 12:818.
[59] Coelho T, Adams D, Silva A, Lozeron P, Hawkins P N, Mant T, et al. Safety and efficacy of RNAi therapy for transthyretin amyloidosis. N Engl J Med. 2013; 369:819-29.
[60] Fitzgerald K, Frank-Kamenetsky M, Shulga-Morskaya S, Liebow A, Bettencourt B R, Sutherland J E, et al. Effect of an RNA interference drug on the synthesis of proprotein convertase subtilisin/kexin type 9 (PCSK9) and the concentration of serum LDL cholesterol in healthy volunteers: a randomised, single-blind, placebo-controlled, phase 1 trial. Lancet. 2014; 383:60-8.
[61] Guarner J, Jernigan J A, Shieh W J, Tatti K, Flannagan L M, Stephens D S, et al. Pathology and pathogenesis of bioterrorism-related inhalational anthrax. Am J Pathol. 2003; 163:701-9.
[62] Kim S S, Ye C, Kumar P, Chiu I, Subramanya S, Wu H, et al. Targeted delivery of siRNA to macrophages for anti-inflammatory treatment. Mol Ther. 2010; 18:993-1001.
[63] Dai Z, Arevalo M T, Li J, Zeng M. Addition of poly (propylene glycol) to multiblock copolymer to optimize siRNA delivery. Bioengineered. 2014; 5:1-8.
[64] Lu Y, Friedman R, Kushner N, Doling A, Thomas L, Touzjian N, et al. Genetically modified anthrax lethal toxin safely delivers whole HIV protein antigens into the cytosol to induce T cell immunity. Proc Natl Acad Sci USA. 2000; 97:8027-32.
[65] Sherer K, Li Y, Cui X, Eichacker P Q. Lethal and edema toxins in the pathogenesis of Bacillus anthraces septic shock: implications for therapy. American journal of respiratory and critical care medicine. 2007; 175:211-21.
[66] Ye C, Choi J G, Abraham S, Shankar P, Manjunath N. Targeting DNA vaccines to myeloid cells using a small peptide. Eur J Immunol. 2014.
[67] Ngundi M M, Meade B D, Lin T L, Tang W J, Burns D L. Comparison of three anthrax toxin neutralization assays. Clin Vaccine Immunol. 2010; 17:895-903.
[68] Young J J, Bromberg-White J L, Zylstra C, Church J T, Boguslawski E, Resau R I, et al. LRPS and LRP6 are not required for protective antigen-mediated internalization or lethality of anthrax lethal toxin. PLoS pathogens. 2007; 3:e27.
[69] Hotchkiss K A, Basile C M, Spring S C, Bonuccelli G, Lisanti M P, Terman B I. TEM8 expression stimulates endothelial cell adhesion and migration by regulating cell-matrix interactions on collagen. Exp Cell Res. 2005; 305:133-44.
[70] Liu S, Zhang Y, Moayeri M, Liu J, Crown D, Fattah R J, et al. Key tissue targets responsible for anthrax-toxin-induced lethality. Nature. 2013; 501:63-8.
[71] Duesbery N S, Webb C P, Leppla S H, Gordon V M, Klimpel K R, Copeland T D, et al. Proteolytic inactivation of MAP-kinase-kinase by anthrax lethal factor. Science. 1998; 280:734-7.
[72] Vitale G, Bernardi L, Napolitani G, Mock M, Montecucco C. Susceptibility of mitogen-activated protein kinase family members to proteolysis by anthrax lethal factor. The Biochemical journal. 2000; 352 Pt 3:739-45.
[73] Moayeri M, Sastalla I, Leppla S H. Anthrax and the inflammasome. Microbes Infect. 2012; 14:392-400.
[74] Levinsohn J L, Newman Z L, Hellmich K A, Fattah R, Getz M A, Liu S, et al. Anthrax lethal factor cleavage of Nlrpl is required for activation of the inflammasome. PLoS pathogens. 2012; 8:e1002638.
[75] Boyden E D, Dietrich W F. Nalplb controls mouse macrophage susceptibility to anthrax lethal toxin. Nature genetics. 2006; 38:240-4.
[76] Leppla S H. Anthrax toxin edema factor: a bacterial adenylate cyclase that increases cyclic AMP concentrations of eukaryotic cells. Proc Natl Acad Sci USA. 1982; 79:3162-6.
[77] Abrami L, Bischofberger M, Kunz B, Groux R, van der Goot F G. Endocytosis of the anthrax toxin is mediated by clathrin, actin and unconventional adaptors. PLoS pathogens. 2010; 6:e1000792.
[78] Abrami L, Kunz B, Deuquet J, Bafico A, Davidson G, van der Goot F G. Functional interactions between anthrax toxin receptors and the WNT signaling protein LRP6. Cellular microbiology. 2008; 10:2509-19.
[79] Hine C M, Seluanov A, Gorbunova V. Rad51 promoter-targeted gene therapy is effective for in vivo visualization and treatment of cancer. Mol Ther. 2012; 20:347-55.
[80] Popov S G, Popova T G, Kashanchi F, Bailey C. Targeting the inflammasome and adenosine type-3 receptors improves outcome of antibiotic therapy in murine anthrax. World journal of biological chemistry. 2011; 2:98-104.
[81] Migone T S, Subramanian G M, Zhong J, Healey L M, Corey A, Devalaraja M, et al. Raxibacumab for the treatment of inhalational anthrax. N Engl J Med. 2009; 361:135-44.
[82] Longest P W, Golshahi L, Behara S R, Tian G, Farkas D R, Hindle M. Efficient Nose-to-Lung (N2L) Aerosol Delivery with a Dry Powder Inhaler. J Aerosol Med Pulm Drug Deliv. 2014.
[83] Tian G, Hindle M, Longest P W. Targeted Lung Delivery of Nasally Administered Aerosols. Aerosol Sci Technol. 2014; 48:434-49.
[84] Longest P W, Walenga R L, Son Y J, Hindle M. High-efficiency generation and delivery of aerosols through nasal cannula during noninvasive ventilation. J Aerosol Med Pulm Drug Deliv. 2013; 26:266-79.
[85] Tian G, Longest P W, Li X, Hindle M. Targeting aerosol deposition to and within the lung airways using excipient enhanced growth. J Aerosol Med Pulm Drug Deliv. 2013; 26:248-65.
[86] Kumar A, Wonganan P, Sandoval M A, Li X, Zhu S, Cui Z. Microneedle-mediated transcutaneous immunization with plasmid DNA coated on cationic PLGA nanoparticles. J Control Release. 2012; 163:230-9.
[87] Ragelle H, Riva R, Vandermeulen G, Naeye B, Pourcelle V, Le Duff C S, et al. Chitosan nanoparticles for siRNA delivery: optimizing formulation to increase stability and efficiency. J Control Release. 2014; 176:54-63.
[88] Niu M, Naguib Y W, Aldayel A M, Shi Y C, Hursting S D, Hersh M A, et al. Biodistribution and in vivo activities of tumor-associated macrophage-targeting nanoparticles incorporated with Doxorubicin. Mol Pharm. 2014; 11:4425-36.
[89] Zhu S, Li X, Lansakara P D, Kumar A, Cui Z. A nanoparticle depot formulation of 4-(N)-stearoyl gemcitabine shows a strong anti-tumour activity. J Pharm Pharmacol. 2013; 65:236-42.
[90] Zhu S, Wonganan P, Lansakara P D, O'Mary H L, Li Y, Cui Z. The effect of the acid-sensitivity of 4-(N)-stearoyl gemcitabine-loaded micelles on drug resistance caused by RRM1 overexpression. Biomaterials. 2013; 34:2327-39.
[91] Li X, Aldayel A M, Cui Z. Aluminum hydroxide nanoparticles show a stronger vaccine adjuvant activity than traditional aluminum hydroxide microparticles. J Control Release. 2014; 173:148-57.
[92] Reeves C, Charles-Horvath P, Kitajewski J. Studies in mice reveal a role for anthrax toxin receptors in matrix metalloproteinase function and extracellular matrix homeostasis. Toxins. 2013; 5:315-26.
[93] Cullen M, Seaman S, Chaudhary A, Yang M Y, Hilton M B, Logsdon D, et al. Host-derived tumor endothelial marker 8 promotes the growth of melanoma. Cancer research. 2009; 69:6021-6.
[94] Chaudhary A, Hilton M B, Seaman S, Haines D C, Stevenson S, Lemotte P K, et al. TEM8/ANTXR1 blockade inhibits pathological angiogenesis and potentiates tumoricidal responses against multiple cancer types. Cancer cell. 2012; 21:212-26.
[95] Liu S, Crown D, Miller-Randolph S, Moayeri M, Wang H, Hu H, et al. Capillary morphogenesis protein-2 is the major receptor mediating lethality of anthrax toxin in vivo. Proc Natl Acad Sci USA. 2009; 106:12424-9.
[96] Reeves C V, Wang X, Charles-Horvath P C, Vink J Y, Borisenko V Y, Young J A, et al. Anthrax toxin receptor 2 functions in ECM homeostasis of the murine reproductive tract and promotes MMP activity. PLoS ONE. 2012; 7:e34862.
[97] Chen Z, Schneerson R, Lovchik J, Lyons C R, Zhao H, Dai Z, et al. Pre- and postexposure protection against virulent anthrax infection in mice by humanized monoclonal antibodies to Bacillus anthraces capsule. Proc Natl Acad Sci USA. 2011; 108:739-44.
[98] AVMA. AVMA Guidelines for the Euthanasia of Animals: 2013 Edition. Schaumburg, Ill. 2013. p. 1-102.

1. A composition for the targeted-delivery of small interference RNA against bacteria comprising:
a detoxified bacterial protein toxin that comprises a highly positively charged region; and
an siRNA that is specific to, and knocks-down expression of one or more genes related to one or more virulence factors of the bacteria, wherein the siRNA is bound to the highly positively charged region of the detoxified bacterial protein toxin.

2. The composition of claim 1, wherein the bacteria is Bacillus anthracis.

3. The composition of claim 1, wherein the composition is adapted for post exposure prophylaxis or therapy.

4. The composition of claim 1, wherein the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2).

5. The composition of claim 1, wherein the detoxified bacterial toxin is selected from at least one of: an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria;
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide;
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA); or
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA).

6. (canceled)

7. (canceled)

8. (canceled)

9. The composition of claim 1, wherein the highly positively charged region comprises 5, 6, 7, 8, 9, 10, 11, 12, 13, 14, 15 or more arginine, lysine, or histidine residues, or combinations thereof.

10. A method of making a therapy against a bacterial infection comprising:
preparing a detoxified bacterial protein toxin that comprises a highly positively charged region; and
binding to the detoxified bacterial protein toxin an siRNA that is specific to, and knocks-down expression of one or more genes related to one or more virulence factors of the bacteria, wherein the siRNA is bound to the highly positively charged region of the detoxified bacterial protein toxin, wherein the detoxified bacterial protein toxin delivers the siRNA to a host cell.

11. The method of claim 10, wherein the bacteria is Bacillus anthracis.

12. The method of claim 10, wherein the composition is adapted for post exposure prophylaxis or therapy.

13. The method of claim 10, wherein the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2).

14. The method of claim 10, wherein the detoxified bacterial toxin is selected from at least one of: an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria;
an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide;
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA); or
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA).

15. (canceled)

16. (canceled)

17. (canceled)

18. The method of claim 10, wherein the highly positively charged region comprises 5, 6, 7, 8, 9, 10, 11, 12, 13, 14, 15 or more arginine, lysine, or histidine residues, or combinations thereof.

19. A composition for the targeted-delivery of small interference RNA against Bacillus anthracis comprising:
a detoxified bacterial protein toxin that is a fusion protein that further comprises a highly positively charged region; and
an siRNA that is specific to, and knocks-down expression of one or more genes related to one or more virulence factors of the Bacillus anthracis, wherein the siRNA is bound to the highly positively charged region of the detoxified bacterial protein toxin.

20. The composition of claim 19, wherein the composition is adapted for post exposure prophylaxis or therapy.

21. The composition of claim 19, wherein the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2).

22. The composition of claim 19, wherein the detoxified bacterial toxin is selected from at least one of: an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria;
an N-fragment of edema factor (EFn), the N-fragment lethal factor (LFn), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide;
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and a protective antigen (PA); or
an N-fragment of edema factor (EFn, the first 254 amino acids of EF), the N-fragment lethal factor (LFn, the first 254 amino acids of LF), and the PA binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and a protective antigen (PA).

23. (canceled)

24. (canceled)

25. (canceled)

26. The composition of claim 19, wherein the highly positively charged region comprises 5, 6, 7, 8, 9, 10, 11, 12, 13, 14, 15 or more arginine, lysine, or histidine residues, or combinations thereof.

27. A method of treating a subject suspected of being infected with a pathogenic bacterial comprising:
preparing a detoxified bacterial protein toxin that comprises a highly positively charged region;
binding to the detoxified bacterial protein toxin an siRNA to form a protein-siRNA complex, wherein the siRNA is specific to, and knocks-down expression of one or more genes related to one or more virulence factors of the bacteria, wherein the siRNA is bound to the highly positively charged region of the detoxified bacterial protein toxin, wherein the detoxified bacterial protein toxin delivers the siRNA to a host cell; and
providing the subject an effective amount of the protein-siRNA complex sufficient to treat the bacterial infection.

28. The method of claim 27, wherein the bacteria is Bacillus anthracis.

29. The method of claim 27, wherein the composition is adapted for post exposure prophylaxis or therapy.

30. The method of claim 27, wherein the virulence factor is selected from anthrax toxin receptor 1 (ANTXR1 or tumor endothelial marker 8/TEM8), or anthrax toxin receptor 2 (ANTXR2 or capillary morphogenesis protein 2/CMG2).

31. The method of claim 27, wherein the detoxified bacterial toxin is selected from at least one of: an N-fragment of edema factor (EFn), the N-fragment lethal factor (LFn), and the protective antigen (PA) binding peptide (Val Tyr Tyr Glu Ile Gly Lys)(SEQ ID NO:3) in EF (residues 136 to 142) or LF (residues 147 to 153) from the bacteria;
an N-fragment of edema factor (EFn), the N-fragment lethal factor (LFn), and the protective antigen (PA) binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide;
an N-fragment of edema factor (EFn), the N-fragment lethal factor (LFn), and the protective antigen (PA) binding peptide (Val Tyr Tyr Glu Ile Gly Lys)(SEQ ID NO:3) in EF (residues 136 to 142) or LF (residues 147 to 153) from the bacteria, are a fusion protein that further comprises an exogenous peptide, and the PA; or
an N-fragment of edema factor (EFn), the N-fragment lethal factor (LFn), and the protective antigen (PA)binding peptide (Val Tyr Tyr Glu Ile Gly Lys)(SEQ ID NO:3) in EF (residues 136 to 142) or LF (residues 147 to 153) from the bacteria, are a fusion protein that further comprises an influenza antigen, and the PA.

32. (canceled)

33. (canceled)

34. (canceled)

35. The method of claim 27, wherein the highly positively charged region comprises 5, 6, 7, 8, 9, 10, 11, 12, 13, 14, 15 or more arginine, lysine, or histidine residues, or combinations thereof.


SEQUENCE LISTING (3 sequences)

SEQ ID NO: 1 - protein, 9 aa, Artificial Sequence
  Synthetic peptide
RRRRRRRRR

SEQ ID NO: 2 - protein, 19 aa, Artificial Sequence
  Synthetic peptide
VYYEIGLGGGRRRRRRRRR

SEQ ID NO: 3 - protein, 7 aa, Artificial Sequence
  Synthetic peptide
VYYEIGK